(19) United States
(12) Patent Application Publication
(10) Pub. No.: US 2016/0082905 A1
(43) Pub. Date: Mar. 24, 2016

(54) SYSTEM AND METHOD FOR ANALYZING FUEL EFFICIENCY BASED ON DRIVING BEHAVIOR

(71) Applicant: Yuan Ze University, Zhongli City (TW)

(72) Inventors: Chia-Yu HSU, New Taipei City (TW); Yi-Ting WANG, Pingtung City (TW); Chin-Sheng YANG, Pingzhen City (TW); Liang-Chih YU, Zhongli City (TW); Pei-Chann CHANG, Zhongli City (TW); Kuo-Hua LAI, Taoyuan City (TW)

(21) Appl. No.: 14/569,192

(22) Filed: Dec. 12, 2014

(30) Foreign Application Priority Data
Sep. 19, 2014 (TW) 103132386

(51) Int. Cl. B60R 16/023; G07C 5/08; B60W 40/12; G01N 33/22

(52) U.S. Cl. CPC B60R 16/0236; G01N 33/22; G07C 5/0808; B60W 40/12; B60W 2560/00

(57) ABSTRACT

A fuel efficiency analysis system is provided, which includes a capture device, an analysis process unit and a transform unit. The capture device captures driving data of automobile which is driven by a driver during at least one driving course, the analysis process unit analyzes the driving data to obtain inefficient driving period, and processes the inefficient driving period to generate a plurality of driving time intervals, and the transform unit transforms the plurality of driving time intervals into a plurality of driving efficiencies, and then the plurality of driving efficiencies is transformed into ODE (overall driving effectiveness). A method for analyzing fuel efficiency is also disclosed.

### CROSS-REFERENCE TO RELATED APPLICATION

This application claims priority from Taiwan Patent Application No. 103132386, filed on Sep. 19, 2014, in the Taiwan Intellectual Property Office, the content of which are hereby incorporated by reference in their entirety for all purposes.

### BACKGROUND OF THE INVENTION

1. Field of the Invention

This invention relates to a system and a method for analyzing fuel efficiency based on driving behavior, and more specifically, to a fuel efficiency analysis system and a method thereof which is to build an evaluation index of an overall driving effectiveness (ODE) by defining reasons of inefficient driving period and consumption during one driving course, such that a driver is able to effectively handle the application of driving time to accomplish the purpose of continuously improving the driving efficiency.

2. Description of the Related Art

Even though there have been diverse researches on Eco-driving and calculation of fuel efficiency, method of comparing fuel efficiency is not objective due to the fuel efficiency is limited by facts of automobile types and environment. For example, imported and exported automobiles may have a difference in the performance of fuel efficiency, and similarly, sport utility vehicles and sedans may also have different performance of fuel efficiency.

In addition, different driving behaviors of a driver may also have direct impact on the fuel efficiency. For instance, a driver driving with a big rush always results in a worse fuel efficiency. However, there is currently no suitable evaluation index for assessing driving efficiency for a driver during a driving course.

As a result, it is in urgent need of having a method for analyzing fuel efficiency based on driving behavior of a driver to analyze the relationship between the driving time and fuel efficiency under the circumstances of various driving behaviors, as well as to establish the technique of evaluation indexes.

### SUMMARY OF THE INVENTION

According to an aspect of the present invention, a fuel efficiency analysis system is provided, which may include a capture device to capture driving data of an automobile which may be driven by a driver, an analysis process unit to analyze the driving data to obtain inefficient driving period, and to process the inefficient driving period to generate a plurality of driving time intervals, and a transform unit to transform the plurality of driving time intervals into a plurality of driving efficiencies, and then to transform the plurality of driving efficiencies into an overall driving effectiveness (ODE).

The driving data may include route information, driving speeds, driving accelerations, throttle depths, an instantaneous fuel efficiency, engine speeds, engine loads and a total automobile operating time during the at least one driving course.

The inefficient driving period may be obtained by analyzing the route information, the driving speed, the driving acceleration, the throttle depths, the instantaneous fuel efficiency, the engine speeds and the engine loads, and the inefficient driving period may include automobile waiting time, automobile actuating time, environment impact time, noncompliance driving speed time and behavior change time, and the automobile waiting time may be a time interval in which the automobile waits and idles during the at least one driving course, an automobile actuating time may be a time interval from a start of an engine of the automobile to a stable speed of the automobile, the environment impact time may be a time interval in which the automobile may be driven through routes of uphills, downhills, corners, and curves by the driver, the noncompliance driving speed time may be a time interval in which a speed of the automobile may be out of a range of a preset driving speed and a load and speed of the engine the automobile may be out of a range of a preset engine load and speed, and the behavior change time may be a time interval in which the automobile may be driven in sudden accelerations and decelerations by the driver.

The plurality of driving time intervals may include an automobile driving time, a normal driving time, straight-line driving time, stable driving time and Eco-driving time, wherein the automobile driving time may be obtained based on the total automobile operating time and the automobile waiting time, the normal driving time may be obtained based on the automobile driving time and the automobile actuating time, the straight-line driving time may be obtained based on the normal driving time and the environment impact time, the stable driving time may be obtained based on the straight-line driving time and the noncompliance driving speed time, and the Eco-driving time may be obtained based on the stable driving time and the behavior change time.

The plurality of driving efficiencies may include an automobile driving efficiency, a normal driving efficiency, a straight-line driving efficiency, a stable driving efficiency and an Eco-driving efficiency, and the overall driving effectiveness may be obtained by multiplying the automobile driving efficiency, the normal driving efficiency, the straight-line driving efficiency, the stable driving efficiency and the Eco-driving efficiency.

According to another aspect of the present invention, a method for analyzing fuel efficiency is provided which may include the following steps. A capture device may be configured to capture driving data of an automobile which is driven by a driver during at least one driving course. An analysis process unit may be configured to analyze the driving data of the driver to obtain inefficient driving period, and to process the inefficient driving period to generate a plurality of driving time intervals. A transform unit may be configured to transform the plurality of driving time intervals into a plurality of driving efficiencies, and then the plurality of driving efficiencies may be transformed into the plurality of driving efficiencies into ODE (overall driving effectiveness).

The driving data may include route information, driving speeds, driving accelerations, throttle depths, an instantaneous fuel efficiency, engine speeds, engine loads and a total automobile operating time.

The inefficient driving period may be obtained by analyzing the route information, the driving speeds, the driving accelerations, the throttle depths, the instantaneous fuel efficiency, the engine speeds and the engine loads, and the inefficient driving period may include automobile waiting time, automobile actuating time, environment impact time, noncompliance driving speed time and behavior change time, and the automobile waiting time may be a time interval in which the automobile waits and idles during the at least one driving course, an automobile actuating time may be a time interval from a start of an engine of the automobile to a stable speed of the automobile, the environment impact time may be a time interval in which the automobile may be driven through routes of uphills, downhills, corners, and curves by the driver, the noncompliance driving speed time may be a time interval in which a speed of the automobile may be out of a range of a preset driving speed and a load and speed of the engine the automobile may be out of a range of a preset engine load and speed, and the behavior change time may be a time interval in which the automobile may be driven in sudden accelerations and decelerations by the driver.

The plurality of driving time intervals may include automobile driving time, normal driving time, straight-line driving time, stable driving time and Eco-driving time, wherein the automobile driving time may be obtained based on the total automobile operating time and the automobile waiting time, the normal driving time may be obtained based on the automobile driving time and the automobile actuating time, the straight-line driving time may be obtained based on the normal driving time and the environment impact time, the stable driving time may be obtained based on the straight-line driving time and the noncompliance driving speed time, and the Eco-driving time may be obtained based on the stable driving time and the behavior change time.

The plurality of driving efficiencies may include an automobile driving efficiency, a normal driving efficiency, a straight-line driving efficiency, a stable driving efficiency and an Eco-driving efficiency, and the overall driving effectiveness may be obtained by multiplying the automobile driving efficiency, the normal driving efficiency, the straight-line driving efficiency, the stable driving efficiency and the Eco-driving efficiency.

After the ODE is obtained, it may further compare the plurality of ODEs obtained in the plurality of driving courses to build the evaluation index for the improvement, wherein the evaluation index may include improvement index of driving behavior and improvement index of driving efficiency.

### BRIEF DESCRIPTION OF THE DRAWINGS

FIG. 1 is a block diagram schematically showing a fuel efficiency analysis system in accordance with an embodiment of the present invention.

FIG. 2 is a flow chart schematically showing analysis process of driving time in accordance with an embodiment of the present invention.

FIG. 3 is a structural diagram of driving time obtained from the analysis of driving time in accordance with the flow chart of FIG. 2 of the present invention.

FIG. 4 is a flow chart schematically showing a method for analyzing fuel efficiency in accordance with an embodiment of the present invention.

### DETAILED DESCRIPTION OF THE PREFERRED EMBODIMENTS

Hereinafter, embodiments of the present invention will be described in detail with reference to the accompanying drawings so that those skilled in the art to which the present invention pertains can realize the present invention. As those skilled in the art would realize, the described embodiments may be modified in various different ways, all without departing from the spirit or scope of the present invention.

In accordance with the embodiment(s) of the present invention, the components, process steps, and/or data structures described herein may be implemented using various types of operating systems, computing platforms, computer programs, and/or general purpose machines. In addition, those of ordinary skill in the art will recognize that devices of a less general purpose nature, such as hardwired devices, field programmable gate arrays (FPGAs), application specific integrated circuits (ASICs), or the like, may also be used without departing from the scope and spirit of the inventive concepts disclosed herein. Where a method comprising a series of process steps is implemented by a computer or a machine and those process steps can be stored as a series of instructions readable by the machine, they may be stored on a tangible medium such as a computer memory device (e.g., ROM (Read Only Memory), PROM (Programmable Read Only Memory), EEPROM (Electrically Erasable Programmable Read Only Memory), FLASH Memory, Jump Drive, and the like), magnetic storage medium (e.g., tape, magnetic disk drive, and the like), optical storage medium (e.g., CD-ROM, DVD-ROM, paper card and paper tape, and the like) and other known types of program memory.

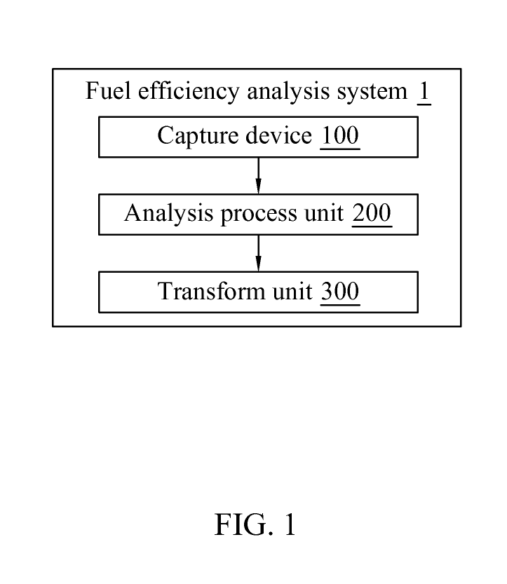

Please refer to FIG. 1, which is a block diagram schematically showing a fuel efficiency analysis system in accordance with an embodiment of the present invention. A fuel efficiency analysis system 1 according to the present invention may include a capture device 100, an analysis process unit 200 and a transform unit 300. For the sake of clearly analyzing each driving parameter of an automobile, capture device 100 may be mounted on an automobile to capture route information, driving speeds, driving accelerations, throttle depths, an instantaneous fuel efficiency, engine speeds, engine loads and a total automobile operating time during one driving course of a driver, and to generate the driving data including the aforementioned information. Analysis process unit 200 may receive the data information obtained from capture device 100 and analyze the driving data to obtain the inefficient driving period. The fuel efficiency analysis system 1, the capture device 100, the analysis process unit 200, and the transform unit 300 may include sensors, circuits, storages, processors, and may be programmed to process, record, calculate, and analyze the aforementioned data and information associated to the automobile and behavior of the driver.

Here, the inefficient driving period may include inefficient driving period defined by a fuel efficiency analysis system 1 in accordance with the present invention, the inefficient driving period may include automobile waiting time, automobile actuating time, environment impact time, noncompliance driving speed time and behavior change time, and definitions thereof may be provided as follows: the automobile waiting time is a time interval in which the automobile waits and idles during the driving course, which also means the time interval generates from the total automobile operating time deducts the automobile driving time, the automobile waiting time includes condition of the idling, in which the automobile stops without turning off the engine such as waiting for the traffic signals, temporary parking and so on. The reason it is mentioned herein is that once the engine of the automobile is actuated, it consumes the fuel even without driving. The automobile actuating time is a time interval from a start of an engine of the automobile to a stable speed of the automobile, and here, situations concerning the unstable speed when actuating the automobile and rapid increase of the engine loads may all cause the efficiency of fuel efficiency to be decreased. The environment impact time is a time interval in which the automobile is driven through routes of uphills, downhills, corners, and curves by the driver, as well as the lower driving efficiency caused by traffic jam and so on. Here, the route information captured by capture device 100 may include the aforementioned information and road condition during the driving course.

Moreover, the noncompliance driving speed time is a time interval in which a speed of the automobile is out of a range of a preset driving speed and a load of the engine the automobile is out of a range of a preset load, in other words, the noncompliance driving speed time also means the time which a driver may drive without complying with the preset speed limit. According to an embodiment of the present invention, the driving routes are divided into two portions based on the driving region, including the routes of highway and ordinary roads. It is regarded as inefficient fuel efficiency under following conditions: 1.) when driving speed is between 80 km/hr and 120 km/hr, and the engine speed is higher than 2400 rpm on highway; 2.) when driving speed is between 30 km/hr and 80 km/hr, the engine speed higher than 2000 rpm, and the engine load is higher than 50% on ordinary roads; 3.) when the driving speed is lower than 30 km/hr and the engine load is higher than 70% on ordinary roads; 4.) the driving speed is higher than 80 km/hr on the ordinary roads; 5.) the driving speed is higher than 120 km/hr and the engine load of higher than 70% on highway. These conditions are all regarded as the inefficient fuel efficiency.

The driving behavior time is a time interval in which the automobile is driven in sudden accelerations and decelerations by the driver means the time which an automobile is driven by a driver with impetuous driving behavior. The impetuous driving behavior includes rush acceleration, overtaking, sudden deceleration and an emergency brake, and when situations concerning that a driver is accelerating in a rush and overtaking, as well as the engine load with higher than 80%, they are served as the inefficient fuel efficiency due to behavior changes. Analysis process unit 200 may analyze the captured driving data according to the aforementioned definitions.

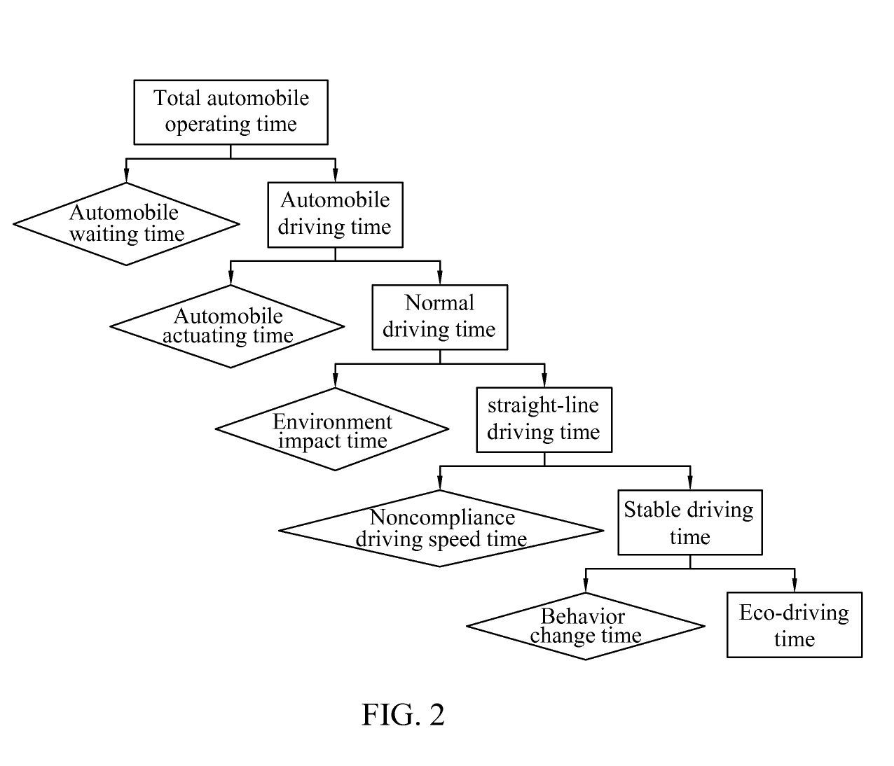

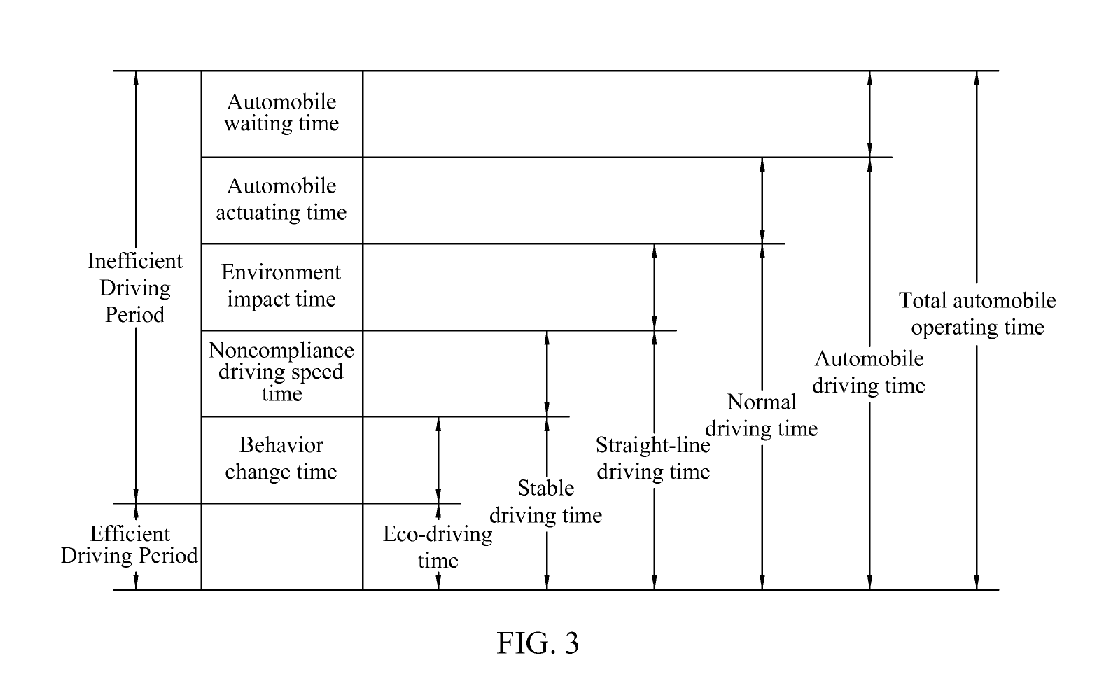

Now please refer to FIG. 2 along with FIG. 3. FIG. 2 is a flow chart schematically showing analysis process of driving time in accordance with an embodiment of the present invention, and FIG. 3 is a structural diagram of driving time obtained from the analysis of driving time in accordance with the flow chart of FIG. 2 of the present invention. The analysis process of driving time will now be described in accordance with exemplary embodiment of the present invention. After the driving data is analyzed by the analysis process unit 200 to obtain the inefficient driving period, the automobile driving time, the normal driving time, straight-line driving time, stable driving time and Eco-driving time may further be obtained subsequently. As shown in FIGS. 2 and 3, the total automobile operating time (Ttotal) captured by the capture device 200 may be divided into efficient driving period and inefficient driving period. That is, the efficient driving period is the time when the fuel efficiency is capable of being applied efficiently, and the inefficient driving period is the time when the fuel efficiency is incapable of being used efficiently when the automobile is operating. The total automobile operating time (Ttotal) is deducted by the aforementioned automobile waiting time to obtain an automobile driving time (Tavaible), and the automobile driving time (Tavaible) may be deducted by the automobile actuating time to obtain the normal driving time (Tnormal) after the preceding automobile actuating time is found. In the automobile actuating time, the automobile is under the unstable conditions while actuating. The normal driving time (Tnormal) may be deducted by the environment impact time to obtain the straight-line driving time (Tline). Namely, the straight-line driving time (Tline) is the time interval of the automobile driving time that not being affected by the driving environment, such as uphills, downhills, corners, and curves. The straight-line driving time (Tline) may be deducted by the aforementioned noncompliance driving speed time to obtain the stable driving time (Tstable). The stable driving time (Tstable) may be deducted by the defined behavior change time to obtain the Eco-driving time (Teconomy), and the analysis result is shown in FIG. 3.

Next, the total automobile operating time (Ttotal), the automobile driving time (Tavaible), the normal driving time (Tnormal), the straight-line driving time (Tline), the stable driving time (Tstable) and the Eco-driving time (Teconomy) analyzed by analysis process unit 200 may further be transformed by a transform unit 300 to obtain an available efficiency (AE), a normal efficiency (NE), a straight-line efficiency (LE), a stable efficiency (SE) and an economy efficiency (EE) according to the following formula:

AE=(Tavaible/Ttotal)×100%

NE=(Tnormal/Tavaible)×100%

LE=(Tline/Tnormal)×100%

SE=(Tstable/Tline)×100%

EE=(Teconomy/Tstable)×100%

Here, AE denotes the proportion of practical driving time in the total automobile operating time. AE can be used to evaluate influences generated from situations related to temporary parking, waiting for the traffic signals and even the automobile stops without turning off the engine during a driving course of the automobile. NE is obtained by deducting the unstable condition when an automobile is operating, and is used to evaluate and so as to improve the driving behavior when an automobile is operating. LE denotes the automobile driving efficiency without being affected by the driving environment. LE may be used to evaluate as well as improve the choice of driving routes. By the calculation of LE, the percentage of being stuck in traffic jam may be decreased, and the routes with less uphill and turns may be chosen. SE denotes the part of the driving time which a driver is driving in compliance with the legal speed limit. SE is used to judge whether a driver maintains a stable driving speed, and may also be used to judge whether passengers have comfortable experience sitting therein. By the calculation of SE, the unnecessary fuel efficiency can be further saved. EE denotes the time which a driver does not perform impetuous driving behavior during the driving course. EE is used to evaluate the time and frequency of the impetuous driving behavior, and may relatively boost the Eco-driving efficiency.

After AE, NE, LE, SE and EE are transformed by transform unit 300, the driving efficiencies may be introduced in the following equation to obtain the overall driving effectiveness (ODE) of an exemplary embodiment in accordance with the present invention. The equation is:

ODE=AE×NE×LE×SE×EE

The evaluation index of ODE provided in the present invention is used on the basis of the practical driving time, and thus it can not only provide the adjustment of efficiency for the driving behavior, but also immediately determine the reason of time and fuel efficiency based on the defined driving behavior and time thereof to improve the bad driving behavior. In addition, according to the definition of ODE index, it can simply calculate and compare the ODE value of each driving data, and the analysis results are as a result used to sort each assessed subject, and further to be adjusted based on the overall driving effectiveness to conduct the follow-up review and improvement.

Another aspect of the present invention relates to a method for analyzing fuel efficiency, wherein details and definitions thereof are akin to that defined in the aforementioned fuel efficiency analysis system. Consequently, the unnecessary redundant descriptions are omitted hereinafter.

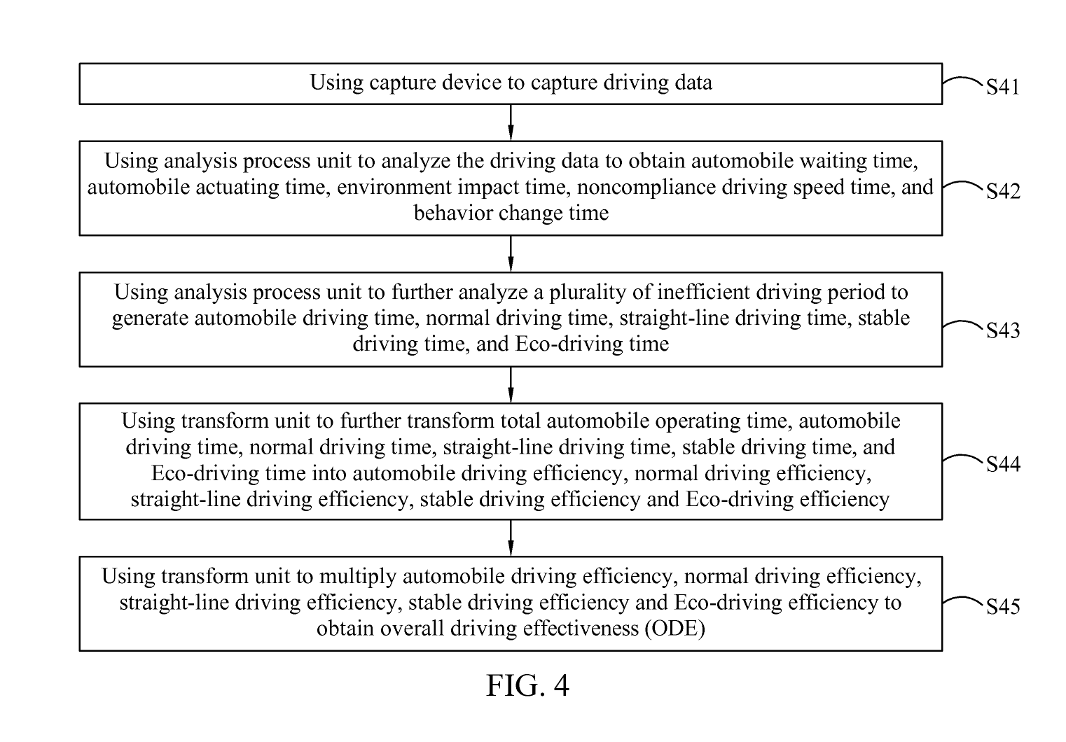

FIG. 4 is a flow chart schematically showing a method for analyzing fuel efficiency in accordance with an embodiment of the present invention.

Please refer to FIG. 4, which demonstrates a method for analyzing fuel efficiency of the present invention having the following steps:

Driving data of an automobile which is driven by a driver during at least one driving course may be captured by a capture device, the driving data may include route information, driving speeds, driving accelerations, throttle depths, an instantaneous fuel efficiency, engine speeds, engine loads and a total automobile operating time (step S41). Moreover, an analysis process unit may receive the driving data concerning the route information, the driving speeds, the driving accelerations, the throttle depths, the instantaneous fuel efficiency, the engine speeds, the engine loads and the total automobile operating time captured by the capture device, and analyze inefficient driving period including automobile waiting time, automobile actuating time, environment impact time, noncompliance driving speed time and behavior change time (step S42). Definitions of the automobile waiting time, the automobile actuating time, the environment impact time, the noncompliance driving speed time and the behavior change time are the same as that defined in the aforementioned description, and therefore the unnecessary details are no longer given herein.

Next, the analysis process unit may further process the inefficient driving period including the automobile waiting time, the automobile actuating time, the environment impact time, the noncompliance driving speed time and the behavior change time to generate a plurality of driving time intervals including automobile driving time, normal driving time, straight-line driving time, stable driving time and Eco-driving time (step S43). Here, the automobile waiting time is a time interval in which the automobile waits and idles during the at least one driving course, an automobile actuating time is a time interval from a start of an engine of the automobile to a stable speed of the automobile, the environment impact time is a time interval in which the automobile is driven through routes of uphills, downhills, corners, and curves by the driver, the noncompliance driving speed time is a time interval in which a speed of the automobile is out of a range of a preset driving speed and a load and speed of the engine the automobile is out of a range of a preset engine load and speed, and the behavior change time is a time interval in which the automobile is driven in sudden accelerations and decelerations by the driver. The total automobile operating time (Ttotal) may be deducted by the aforementioned automobile waiting time to obtain an automobile driving time (Tavaible), and the automobile driving time (Tavaible) may be deducted by the automobile actuating time to obtain the normal driving time (Tnormal). In the automobile actuating time, the automobile is under the unstable condition while actuating. Namely, the normal driving time (Tnormal) may be obtained after the automobile actuating time is found. The normal driving time (Tnormal) may be deducted by the environment impact to obtain the straight-line driving time (Tline), that is the time when the automobile is not being affected by the driving environment during the driving course. The stable driving time (Tstable) may be obtained by deducting the aforementioned noncompliance driving speed time from the straight-line driving time (Tline). The stable driving time (Tstable) may be deducted the defined behavior change time to obtain the Eco-driving time (Teconomy), and the analysis result is shown in FIG. 3.

Subsequently, the Ttotal, the Tavailable, the Tnormal, the Tline, the Tstable and the Teconomy may further be transformed into the AE, the NE, the LE, the SE and the EE by a transform unit (step S44). The transformation by the transform unit is completed on the basis of the preceding definitions thereof, and the unnecessary details are therefore no longer given herein.

Finally, after the transform unit transforming the AE, the NE, the LE, the SE and the EE, it may further multiply the preceding driving efficiencies to obtain the ODE provided in the exemplary embodiment in accordance with the present invention (step S45). The formula is completed on the basis of the preceding definitions thereof, and the unnecessary redundant descriptions are therefore no longer given herein.

Calculation Example:

The following calculation is taken as an example for explaining a method for analyzing fuel efficiency in accordance with the present invention. Assuming that initial Ttotal is set as 2696 seconds. Because Ttotal has to deduct the outlier of 59 seconds with 0 fuel efficiency in advance, and the correct Ttotal is 2696−59=2634 seconds. The automobile waiting time is 739 seconds, and the Tavailable is 2634−739=1895 seconds, and the automobile actuating time is 103 seconds, and Tnormal is 1895−103=1792 seconds. In addition, the environment impact time including time intervals for driving through curves, corners, traffic jam, uphill and downhill on the routes and so on is 19 seconds, and thus Tline is 1792−19=1773 seconds. As each roads have different speed limits, the summation of noncompliance driving speed time is 334 seconds, and Tstable is 1773−334=1439 seconds. Finally, the impetuous driving behavior is inevitable, the Eco-driving time is therefore deducted by the driving behavior change time, such as accelerating or overtaking by 49 seconds, and Teconomy is 1439−49=1387 seconds.

The example of the total automobile operating time is 2693 seconds. When each of the efficient driving period and the inefficient driving period is calculated, proportion of respective efficiency index and ODE is shown as follows:

AE=1895/2634×100%=71.9%

NE=1792/1895×100%=94.6%

LE=1773/1792×100%=98.9%

SE=1439/1773×100%=81.2%

EE=1387/1439×100%=96.4%

ODE=71.9%×94.6%×98.9%×81.2%×96.4%=52.7%

Consequently, a fuel efficiency analysis system in accordance with an embodiment of the present invention may use data mining algorithm to build a fuel efficiency prediction model through a model tree to provide an estimation of fuel efficiency to each driving behavior, and to develop a technique for analyzing the fuel efficiency towards driving behavior, as well as to build an analysis model of evaluation index of the ODE so as to clearly define the efficient driving period and the inefficient driving period for fuel efficiency during at least one driving course.

In conclusion, the evaluation index of the ODE calculated in accordance with embodiments of the present invention is able to simply calculate and compare the ODE value of each driving data, and as a result, the analysis results can not only assess and sort each driving process, but also adjust the results on the basis of the proportion of effectiveness to conduct the follow-up review and improvement. A system and a method for analyzing fuel efficiency based on driving behavior of the present invention are able to analyze the shortcomings so as to improve the driving behavior and driving efficiency according to user's need. In addition, the ODE analyzes the inefficient driving period of the driving time systematically to enable the driver not only being able to handle application of the driving time, but also continuously improving efficiency of the driving time so as to boost the overall driving effectiveness.

Although the present invention has been disclosed in the preceding descriptions, it is not used to limit the present invention. Any person skilled in the art is able to modify and retouch it without departing from the scope and spirit of the invention. Therefore, the protected scope of the present invention is defined on the basis of the following claims.

While the means of specific embodiments in present invention has been described by reference drawings, numerous modifications and variations could be made thereto by those skilled in the art without departing from the scope and spirit of the invention set forth in the claims. The modifications and variations should in a range limited by the specification of the present invention.

What is claimed is:

1. A fuel efficiency analysis system, comprising:
a capture device configured to capture driving data of an automobile which is driven by a driver during at least one driving course;
an analysis process unit configured to analyze the driving data to obtain inefficient driving period, and to process the inefficient driving period to generate a plurality of driving time intervals, and
a transform unit configured to transform the plurality of driving time intervals into a plurality of driving efficiencies, and then to transform the plurality of driving efficiencies into ODE (overall driving effectiveness).

2. The fuel efficiency analysis system of claim 1, wherein the driving data comprises route information, driving speeds, driving accelerations, throttle depths, an instantaneous fuel efficiency, engine speeds, engine loads and a total automobile operating time during the at least one driving course.

3. The fuel efficiency analysis system of claim 2, wherein the inefficient driving period is obtained by analyzing the route information, the driving speeds, the driving accelerations, the throttle depths, the instantaneous fuel efficiency, the engine speeds and the engine loads, and the inefficient driving period comprise automobile waiting time, automobile actuating time, environment impact time, noncompliance driving speed time and behavior change time.

4. The fuel efficiency analysis system of claim 3, wherein the plurality of driving time intervals comprises automobile driving time, normal driving time, straight-line driving time, stable driving time and Eco-driving time, wherein the automobile driving time is obtained based on total automobile operating time and the automobile waiting time, the normal driving time is obtained based on the automobile driving time and the automobile actuating time, the straight-line driving time is obtained based on the normal driving time and the environment impact time, the stable driving time is obtained based on the straight-line driving time and the noncompliance driving speed time, and the Eco-driving time is obtained based on the stable driving time and the behavior change time.

5. The fuel efficiency analysis system of claim 4, wherein the plurality of driving efficiencies comprises an automobile driving efficiency, a normal driving efficiency, a straight-line driving efficiency, a stable driving efficiency and an Eco-driving efficiency, and the overall driving effectiveness is obtained by multiplying the automobile driving efficiency, the normal driving efficiency, the straight-line driving efficiency, the stable driving efficiency and the Eco-driving efficiency.

6. A method for analyzing fuel efficiency, comprising the following steps:
using a capture device to capture driving data of an automobile which is driven by a driver during at least one driving course;
using an analysis process unit to analyze the driving data of the driver to obtain inefficient driving period, and to process the inefficient driving period to generate a plurality of driving time intervals, and
using a transform unit to transform the plurality of driving time intervals into a plurality of driving efficiencies, and then to transform the plurality of driving efficiencies into ODE (overall driving effectiveness).

7. The method for analyzing fuel efficiency of claim 6, wherein the driving data comprises route information, driving speeds, driving accelerations, throttle depths, an instantaneous fuel efficiency, engine speeds, engine loads and total automobile operating time during the at least one driving course.

8. The method for analyzing fuel efficiency of claim 7, wherein the inefficient driving period is obtained by analyzing the route information, the driving speeds, the driving accelerations, the throttle depths, the instantaneous fuel efficiency, the engine speeds and the engine loads, and the inefficient driving period comprises automobile waiting time, automobile actuating time, environment impact time, noncompliance driving speed time and behavior change time.

9. The method for analyzing fuel efficiency of claim 8, wherein the plurality of driving time intervals comprises automobile driving time, normal driving time, straight-line driving time, stable driving time and Eco-driving time, wherein the automobile driving time is obtained based on the total automobile operating time and the automobile waiting time, the normal driving time is obtained based on the automobile driving time and the automobile actuating time, the straight-line driving time is obtained based on the normal driving time and the environment impact time, the stable driving time is obtained based on the straight-line driving time and the noncompliance driving speed time, and the Eco-driving time is obtained based on the stable driving time and the behavior change time.

10. The method for analyzing fuel efficiency of claim 9, wherein the plurality of driving efficiencies comprises an automobile driving efficiency, a normal driving efficiency, a straight-line driving efficiency, a stable driving efficiency and an Eco-driving efficiency, and the overall driving effectiveness is obtained by multiplying the automobile driving efficiency, the normal driving efficiency, the straight-line driving efficiency, the stable driving effectiveness and the Eco-driving efficiency.
